(19) United States
(12) Patent Application Publication
(10) Pub. No.: US 2011/0150851 A1
(43) Pub. Date: Jun. 23, 2011

(54) NUTRITIONAL COMPOSITION FOR INFANTS DELIVERED VIA CAESAREAN SECTION

(75) Inventors: Joachim Schmitt, Hoesbach (DE); Francis Lecroix, Godewaersvelde (FR); Pierre Jesenne, Boeschepe (FR); Bernd Stahl, Rosbach-Rodheim (DE); Günther Boehm, Echzell (DE); Emmanuel Perrin, Boeschepe (FR)

(73) Assignee: N.V. NUTRICIA, Zoetermeer (NL)

(21) Appl. No.: 13/001,991

(22) PCT Filed: Jun. 19, 2009
(86) PCT No.: PCT/NL2009/050364

§ 371 Date: Feb. 11, 2011

(30) Foreign Application Priority Data
Jun. 30, 2008 (NL) PCT/NL2008/050434

(51) Int. Cl. A61K 35/74; A61P 31/04

(52) U.S. Cl. 424/93.44; 424/93.4; 424/93.45

(57) ABSTRACT

The present invention relates to compositions to be administered to infants delivered via caesarean section and in particular to a product comprising inactivated cells and/or bacterial cell fragments of Gram-negative bacteria and of optionally inactivated Gram-positive bacteria. Thereby it is possible to stimulate a fast colonisation of the intestinal microbiota of said infants.

### FIELD OF THE INVENTION

The present invention relates to methods for feeding infants delivered via caesarean section and to compositions to be administered to infants delivered via caesarean section.

### BACKGROUND OF THE INVENTION

Before birth the intestinal tract of the infant is normally sterile. During vaginal delivery the intestinal tract of the infant is inoculated with vaginal and/or faecal bacteria of the mother, resulting in a colonization of the infant's gastrointestinal tract by bacteria originating from the mother. A maternally derived healthy intestinal microbiota has numerous positive effects on the infant, such as a reduced incidence of infections and a strengthened immune system. Negele et al, 2004, Pediatr. Allergy

Immunol. 15: 48-54 disclose that caesarean section infants have an increased risk for wheezing and allergic sensitization.

In infants delivered via caesarean section the colonization by intestinal bacteria is delayed and occurs by bacteria present in the hospital environment, resulting in the development of a different, less optimal intestinal microbiota. The intestinal microbiota of caesarean delivered infants comprises less bacteria, less beneficial bacteria and less species of beneficial bacteria, compared to intestinal microbiota of infants born via the vaginal route. In particular, the profile and content of lactic acid producing bacteria such as Bifidobacterium species of the microbiota of infants delivered via caesarean section is different from the intestinal profile and content of Bifidobacterium species of infants delivered via the vaginal route. Also the amount of Bacteroides species is less and the colonisation with Bacteroides is delayed in caesarean delivered infants. These differences in microbiota persist well into childhood. Grönlund et al, 1999, JPGN 28:19-25, disclose that there are differences in intestinal flora after caesarean delivery compared to vaginally born infants.

Infant formulae are normally designed to mimic the development of an intestinal microbiota in an infant receiving human breast milk, with the implication that all infants react similar to human breast milk and infant formula. However, the sub-population of infants delivered via caesarean section will react differently because the colonization is delayed and less optimal.

WO 2007/045502 discloses the use of at least two different microorganisms, or at least one microorganism and at least one indigestible oligosaccharide or at least two different Bifidobacterium species, subspecies or strains for the manufacture of a composition for enteral administration to an infant delivered via caesarean section. WO 2007/046698 discloses the use of a composition comprising non-digestible oligosaccharide for the manufacture of a composition for enteral administration to an infant delivered via caesarean section. Natren® produces the probiotic product Life Start® which is designed specifically for infants and suitable for infants delivered via caesarean section. Life Start® is made with Bifidobacterium infantis.

Heyman et al, 2005, Acta Paediatrica 94:34-36, disclose the use of non-live micro-organisms in fermented infant formula intended for healthy infants with a positive effect on intestinal function.

US 2006/018890 is directed to the use of treating respiratory infections and acute otitis media in infants by administering bifidobacteria and an adherence promoting strain. Living bacteria are preferred and the experimental data shown use living Bifidobacterium lactis and Lactobacillus GG.

EP 1364586 discloses the use of Lactobacillus paracasei and Bifidobacterium lactis to promote oral tolerance. Optionally the bacteria are dead. Optionally these bacteria are added to fermented products not including infant formula.

Kirjavainen et al, 2003, JPGN 36:223-227, disclose that using viable, but not heat inactivated LGG is a potential approach for the management of atopic eczema and cow's milk allergy.

EP 1597978 discloses a synergistic effect between polyfructose and galacto-oligosaccharide when fermented by infant's faeces.

US 2006/0233773 discloses the use of Lactobacillus GG for preventing or reducing the development of respiratory allergies. These bacteria are living.

Mcvay et al, 2008, J Pediatr Surg 43:25-29, disclose that a formula with live probiotic bacteria was superior compared with chemically acidified, Lactococcis lactis fermented infant formula and control infant formula in reducing pulmonary and gastrointestinal bacterial colonization. The formulae were tested in rabbit pups delivered via caesarean section.

### SUMMARY OF THE INVENTION

Animal experiments showed that already within two hours after birth the intestine of a vaginally born infant shows an immunological response to bacteria, whereas in caesarean section delivered infants no such fast immunological response is observed. This response is initiated by the immunogenic factors of the bacteria and is indicative for tolerance induction against these bacteria, thereby enabling a fast colonisation of the gut. It is believed that this fast immunological response is very important for a healthy development of the infant. Hence, it is particularly desirable to have similar effects in infants born via caesarean section, particularly the tolerance induction and fast colonisation of the gut by bacteria.

The inventors recognised that early exposure of the intestine of the newborn infant that was delivered by caesarean section, to an enteral product which comprises inactivated Gram-negative bacteria and/or bacterial cell fragments of Gram-negative bacteria induces an intestinal tolerance for these Gram-negative bacteria similar to that of vaginally born infants, enabling a fast colonisation of the intestine as in vaginally born infants. Preferably the composition also comprises Gram-positive bacteria, and preferably the Gram-positive bacteria are inactivated, and/or bacterial cell fragments of Gram-positive bacteria. The presence of inactivated cells and/or bacterial cell fragments of both Gram-negative as well as Gram-positive bacteria will advantageously result in an intestinal tolerance to both types of bacteria. The inventors recognized that not only tolerance induction against Gram-positive bacteria is of importance, but also tolerance against Gram-negative bacteria. This is against the prejudice in the field, where Gram-positive bacteria, in particular Bifidobacteria and Lactobacilli are considered to be important. In caesarean section delivered infants a lower amount of Gram-negative bacteria, in particular E. coli and/or Bacteroides was observed, compared to vaginally born infants.

Because the intestinal microbiota plays a crucial role in the development of the infant, in particularly in the stimulation of the immune system, susceptibility for atopic diseases and resistance against infections, it is of utmost importance to stimulate a fast and healthy development of the intestinal microbiota of infants born via caesarean section.

Caesarean section delivered infants are delivered in a hospital environment, which is a risk for pathogenic infection and/or diarrhoea due to the occurrence of nosocomial bacteria. Additionally, the impaired development of a healthy intestinal flora results in faster colonisation of pathogenic bacteria compared to a situation where the infants intestinal tract is inoculated by maternal bacteria. The present invention particularly aims to provide a composition which decreases the incidence and severity of infections and/or diarrhoea in infants born via caesarean section, by inducing tolerance of the infant's gut for beneficial bacteria, by stimulating the growth of beneficial bacteria, preferably lactic acid producing bacteria, and/or by decreasing the growth of adverse bacteria. Hence the present composition can be advantageously used to treat and/or prevent infections in infants born via caesarean section.

Caesarean section delivered infants have an increased risk in atopic diseases such as food allergy, asthma, atopic dermatitis, and/or allergic rhinitis. The present invention particularly aims to provide a composition which decreases the incidence and severity of atopic diseases such as atopic eczema (or atopic dermatitis), allergy and/or asthma in infants born via caesarean section, by improving the intestinal colonization of beneficial bacteria. Hence the present invention can be advantageously used to treat and/or prevent atopic diseases in infants.

The inactivated Gram-negative bacteria and/or cell fragments of Gram-negative bacteria and also optionally Gram-positive bacteria, which are preferably inactivated, and/or cell fragments of Gram-positive bacteria comprise glycoproteins, glycolipids, peptidoglycan, lipopolysaccharides (LPS), lipoteichoic acid (LTA), flagellae, lipoproteins, capsular polysaccharides and/or DNA. Without wishing to be bound by theory, it is believed by the present inventors that these immunogenic molecules induce the tolerance of the intestinal tract against colonisation with Gram-negative and preferably also Gram-positive bacteria. Furthermore, the presence of inactivated Gram-negative bacteria and/or cell fragments from Gram-negative bacteria additionally has the advantage of resulting in an increased immunity against infections, intestinal infections as well as systemic infections, with Gram-negative bacteria. Induction of tolerance against bacteria in the intestinal tract results in a faster colonisation by the desired bacteria, while on the other hand the absence of living cells in the product results in an increased safety and improved product technological properties. The safety advantage is especially important in case of caesarean section delivered infants, which are more vulnerable to infections. The safety advantage is especially important in case of Gram-negative bacteria, which are generally not recognized as safe.

### DETAILED DESCRIPTION OF THE PREFFERED EMBODIMENTS

The present invention concerns a method for providing nutrition to an infant delivered via caesarean section, said method comprising administering to said infant a nutritional composition comprising inactivated Gram-negative bacteria and/or bacterial cell fragments of Gram-negative bacteria and optionally Gram-positive bacteria and/or bacterial cell fragments of Gram-positive bacteria, wherein the composition comprises less than 10³ cfu Gram-negative bacteria per g dry weight of the composition.

In other words the invention concerns a nutritional composition comprising inactivated Gram-negative bacteria and/or bacterial cell fragments of Gram-negative bacteria and optionally Gram-positive bacteria and/or bacterial cell fragments of Gram-positive bacteria, wherein the composition comprises less than 10³ cfu Gram-negative bacteria per g dry weight of the composition for use in providing nutrition to an infant delivered via caesarean section.

The invention can also be worded as the use of a composition comprising inactivated Gram-negative bacteria and/or bacterial cell fragments of Gram-negative bacteria and optionally Gram-positive bacteria and/or bacterial cell fragments of Gram-positive bacteria, wherein the composition comprises less than 10³ cfu Gram-negative bacteria per g dry weight of the composition for the manufacture of a nutritional composition for providing nutrition to an infant delivered via caesarean section.

The invention also concerns a nutritional composition comprising inactivated Gram-negative bacteria and/or bacterial cell fragments of Gram-negative bacteria and optionally Gram-positive bacteria and/or bacterial cell fragments of Gram-positive bacteria, wherein the composition comprises less than 10³ cfu Gram-negative bacteria per g dry weight of the composition.

Wherever herein below reference is made to the nutritional composition of the present invention or further or preferred embodiments of the nutritional composition of the present invention are specified, this is also applicable to the use according to the present invention.

### Caesarean Section

The present invention relates to the enteral administration of a nutritional composition comprising inactivated Gram-negative bacteria and/or bacterial cell fragments of Gram-negative and optionally Gram-positive species to infants delivered via caesarean section. A caesarean section (c-section) is a surgical procedure where an infant is delivered through an incision made in the mother's abdominal wall, and then through the wall of the uterus. A caesarean section is usually performed when it is safer for the mother or the infant than a vaginal delivery. Alternatively, a woman may choose to have a caesarean section rather than deliver her infant vaginally. Throughout this description the terms ‘delivered via caesarean section’, ‘born via caesarean section’, ‘caesarean section delivered’ and ‘caesarean delivered’ etc, are used interchangeably.

Inactivated Bacteria and/or Bacterial Cell Fragments.

The enteral nutritional composition of the present invention comprises inactivated bacteria and/or bacterial cell fragments. Examples of bacterial cell fragments are glycoproteins, glycolipids, peptidoglycan, lipopolysaccharides (LPS), lipoteichoic acid (LTA), flagellae, lipoproteins, capsular polysaccharides and/or DNA. The composition of the present invention comprises inactivated Gram-negative bacteria and/or bacterial cell fragments of Gram-negative bacteria and optionally Gram-positive bacteria. This will induce tolerance to Gram-negative and preferably also Gram-positive bacteria. Preferably inactivated cells and/or bacterial cell fragments of both Gram-negative and Gram-positive bacteria are present, since both are important classes of bacteria colonizing in the intestine of infants. Furthermore, the presence of inactivated Gram-negative bacteria and/or cell fragments from Gram-negative bacteria additionally has the advantage of resulting in an increased immunity against infections, intestinal infections as well as systemic infections, with Gram-negative bacteria.

Gram-negative bacteria are those bacteria that do not retain crystal violet dye in the Gram staining protocol, because of the low amount of peptidoglycan in the cell wall. Gram-negative bacteria have a cytoplasmic membrane and also an outer membrane which comprises LPS. Between these two membranes a thin peptidoglycan layer is present. No teichoic acids or lipoteichoic acids are present. Gram-negative bacteria comprise amongst others the groups of proteobacteria and bacteroides

The inactivated Gram-negative bacteria and/or cell fragments derived thereof used according to the present invention are preferably selected from the group consisting of Bacteroides, Escherichia, Prevotella, Enterobacter, Klebsiella, Proteus, Citrobacter, Pseudomonas, Veillonella, Acinetobacter, and Peptostreptococcus, more preferably Bacteroides. Preferably the Gram-negative bacteria used according to the present invention comprise at least one Bacteroides selected from the group consisting of B. fragilis, B. thetaiotamicron, B. vulgatis B. distasonis, B. ovatus and B. uniformis,more preferably B. fragilis. The colonisation of Bacteroides species, an important group of the intestinal microbiota in infants, is severely delayed in Caesarean delivered infants, especially the colonisation of B. fragilis.

Gram-positive bacteria are bacteria that are stained positive, dark blue or violet, by Gram staining. Gram-positive organisms are able to retain the crystal violet stain because of the high amount of peptidoglycan in the cell wall. Gram-positive bacteria only have one cytoplasmic membrane and lack an outer membrane found as found in Gram-negative bacteria. The Gram-positive bacteria have a thick peptidoglycan layer comprising teichoic acids and lipoteichoic acids. Gram-positive bacteria comprise the groups firmicutes and actinobacteria. The preferably inactivated Gram-positive bacteria and/or cell fragments derived thereof of the present invention are preferably lactic acid bacteria, more preferably bacteria of the genus Lactobacillus and/or Bifidobacterium and/or Streptococcus. Preferably the Gram-positive bacteria used according to the present invention comprise at least one Bifidobacterium selected from the group consisting of B.breve, B.infantis, B.bifidum, B.catenulatum, B.adolescentis, B.thermophilum, B.gallicum, B.animalis or lactis, B.angulatum, B.pseudocatenulatum, B. thermacidophilum and B.longum more preferably B. breve, B. infantis, B. bifidum, B. catenulatum, B. longum, more preferably B. longum and B. breve, even more preferably B. breve, most preferably B. breve 1-2219 deposited at the CNCM in Paris, France. Preferably the lactic acid bacteria used according to the present invention comprise at least one, more preferably at least two, even more preferably at least three, most preferably at least four different Bifidobacterium species. Preferably the lactic acid bacteria used comprise at least B. longum and B. breve. The above-mentioned combinations commonly aim to increase the tolerance against a more diverse quantity of Gram-positive bacteria in the intestine of the caesarean section delivered infant. This beneficially affects the infant, proving numerous health benefits.

Preferably the Gram-positive bacteria used according to the present invention comprise at least one, more preferably at least two Lactobacillus species selected from the group consisting of L. casei, L. reuteri, L paracasei, L. rhamnosus, L. acidophilus, L. johnsonii, L. lactis, L. salivarius, L. crispatus, L. gasseri, L. zeae, L. fermentum and L. plantarum, more preferably L. casei, L. paracasei, L. rhamnosus, L. johnsonii, L. acidophilus, L. fermentum and even more preferably L. paracasei. Even more preferably the lactic acid bacteria used according to the present invention comprise Bifidobacterium breve and/or Lactobacillus paracasei, because the growth of these bacteria in impaired in the intestine of formula fed infants—even when non-digestible oligosaccharides are added to the infant formula—compared to the intestine of breast fed infants. The further increased biodiversity will have a stimulatory effect on health of the newborn delivered by caesarean section.

Preferably the Gram-positive bacteria used comprise at least one microorganism selected from the group consisting of Carnobacterium, Enterococcus, Lactococcus, Leuconostoc, Oenococcus, Pediococcus, Streptococcus, Tetragenococcus, Vagococcus and Weissella, more preferably Streptococcus thermophilus. Preferably the Gram-positive bacteria used according to the present invention comprise at least one microorganism selected from the group consisting of Ruminococcus, Eubacterium and Propionibacterium, more preferably the group consisting of R. bromii, R. obeum, R callidus, E. rectale, E lentum, E aerofaciens, and P. freudenreichii. The further increased biodiversity will have a stimulatory effect on health of the newborn delivered by Caesarean section.

### Methods of Inactivation or Fragmentation of Bacteria

Methods for obtaining a biomass of living cell bacterial cells are known in the art. Subsequently, the living cells of Gram-negative and preferably also Gram-positive bacteria are essentially all eliminated, for example by inactivation and/or physical removal. The cells are preferably inactivated. Living bacterial cells are preferably inactivated by methods selected from the group consisting of heat treatment, UV treatment, sonication, treatment with oxygen, treatment with bactericidals such as ethanol, ultra high pressure application, high pressure homogenisation and/or use of a cell disruptor. Preferably the bacteria are heat killed. Preferable ways of heat killing are pasteurization, sterilization, ultra high temperature treatment, spray cooking and/or spray drying at temperatures bacteria do not survive. Cell fragments are preferably obtained by heat treatment, sonication, treatment with bactericidals such as ethanol, ultra high pressure application, high pressure homogenisation and/or use of a cell disruptor. Preferably intact cells of bacteria are removed by physical elimination such as filtration or centrifugation, for example centrifugation at 1 h at 3000 g, with the intact cells remaining in the pellet or retentate and the cell fragments remaining in the supernatant and/or filtrate, respectively. The inactivation and/or physical removal of living cells is such that the amount of living bacteria is below detection limit as used by conventional plating techniques known in the art. This detection limit is less than 10³ cfu living cells, preferably less than 10² cfu living cells. Preferably the composition comprises less than 10³ cfu living cells of Gram-negative bacteria, more preferably less than 10³ cfu living cells of total live bacteria, based on g dry weight composition. More preferably the composition comprises less than 10² cfu living cells of Gram-negative bacteria, more preferably less than 10² cfu living cells of total live bacteria, based on g dry weight composition. Molecular techniques such as real time PCR techniques as indicated below for detection of inactivated bacterial cells and/or bacterial cell fragments are not applicable, since they are not indicative for living bacteria.

In one embodiment the present nutritional composition comprises substantially no living Gram-negative bacteria and preferably no living Gram-positive bacteria as well. The term “substantially no living bacteria” means that the amount of living bacteria is below the detection limit of conventional plating techniques known in the art.

Inactivation of living cells has the advantage that, after production, the final nutritional composition can be pasteurised and/or sterilised, consequently reducing the chance of contamination with harmful micro-organisms, such as E. sakazakii. This is especially of importance for caesarean delivered infants since due to their delayed intestinal colonisation they are more prone to infections. So the present invention enables liquid, ready-to-use formula to be prepared and stored at room temperature. Furthermore, the dose of bioactive components received by each infant and/or toddler can be more easily controlled, since no further growth in a liquid product occurs, nor growth in the intestinal tract of the infant. The latter is a variable factor depending on the individual's intestinal environment, and thereby leads to variations in the extent of beneficial effects in individual infants.

Additional advantages are that the nutritional composition can be stored more easily and with reduced costs, since no special precautions have to be taken to maintain the viability of bacteria at an acceptable level. This is especially the case in products with a water activity above 0.3. Also no post-acidification occurs in stored products with a high water activity and/or in infant formula in the period after reconstitution with water and before consumption. Adverse effect relating to coagulation of proteins and adverse taste are avoided in this way.

Preferred methods for preparing the bacterial cell fragments or inactivated cells of Gram-positive bacteria are disclosed in WO 01/01785, more particular in example 1 and 2 and in WO 2004/093899, more particularly in example 1. Similar methods can be used mutatis mutandis to obtain inactivated cells and/or bacteria cell fragments from Gram-negative bacteria.

### Products Comprising Inactivated Cells or Cell Fragments of Gram-Negative Bacteria and Optionally Gram-Positive Bacteria

Preferably, the present infant and/or toddler nutrition is a fermented composition. Preferably the present nutrition comprises a milk-derived product fermented by Gram-positive lactic acid producing bacteria, more preferably bifidobacteria, lactobacilli and/or streptococci, even more preferably Bifidobacterium breve, Lactobacillus paracasei, and/or Streptococcus thermophilus, of which the cells are inactivated after fermentation. Upon fermentation and/or other interactions of lactic acid producing bacteria with the milk-derived products, additional bioactive compounds may be formed, such as bioactive peptides and/or oligosaccharides, which also stimulate the immune system and/or stimulate the colonization of the intestinal microbiota. The milk derived product is preferably selected from the group consisting of milk, casein, casein protein, casein protein hydrolysate, casein peptides, whey, whey protein, whey protein hydrolysate, whey peptides, and lactose or mixtures thereof. Milk can be whole milk, semi-skimmed milk and/or skimmed milk. Whey can be sweet whey, and/or acid whey. Preferably the composition to be fermented is skimmed milk.

The incubation time for fermentation is preferably at least 2 h, preferably between 4 and 48 h, more preferably between 6 and 24 h, even more preferably between 6 and 12 h. A sufficient long time enables the fermentation and the concomitant production of immunogenic cell fragments such as glycoproteins, glycolipids, peptidoglycan, lipoteichoic acid (LTA), flagellae, lipoproteins, DNA and/or capsular polysaccharides to take place to a high extent, whereas the incubation time need not be unnecessarily long for economical reasons.

Preferably, a milk substrate, preferably skimmed milk, is pasteurized, cooled and fermented with one or more Lactobacillus strains to a certain degree of acidity, upon which the fermented product is cooled and stored. Preferably a second milk-derived product is prepared in a similar way using one or more Bifidobacterium species for fermentation instead. Subsequently, the two fermented products are preferably mixed together and mixed with other components making up an infant formula, except the fat component. Preferably, the mixture is preheated, and subsequently fat is added, homogenized, pasteurized and dried.

Preferably, a milk substrate, preferably lactose, is pasteurized, cooled and fermented with one or more Streptococcus thermophilus strains, upon which the fermented product is cooled and stored. Preferably a second milk-derived product is prepared in a similar way using skimmed milk and one or more Bifidobacterium species for fermentation instead. Subsequently, the two fermented products are preferably mixed together and mixed with other components making up an infant formula, pasteurized and dried.

The inactivated bacteria or cell fragments are preferably obtained from more than 1×10² cfu, in particular at least 1×10³ cfu, Gram-positive plus Gram-negative bacteria, more preferably Gram-negative bacteria, per g based on dry weight of the final composition, more preferably more than 1×10⁴ cfu, even more preferably more than 1×10⁶ cfu. Preferably the inactivated bacteria or cell fragments are obtained from less than 1×10¹¹ cfu Gram-positive and Gram-negative bacteria, more preferably Gram-negative bacteria, per g based on dry weight of the final composition, more preferably 1×10¹⁰ cfu, even more preferably 1×10⁹ cfu. The amount of cfu per g dry weight can be determined in a composition just before the inactivation step. Alternatively, the amount of inactivated bacteria and/or bacterial cell fragments can be determined by real time PCR techniques. For example, the amount of total inactivated or fragmented bacteria can be determined using universal bacterial probes and primers according to Nadkarni et al, 2002, Microbiology 148:257-266. Methods using probes and primers specific for Gram-negative or Gram-positive groups of bacteria can be applied mutatis mutandis.

### Non-Digestible Oligosaccharides

The present composition preferably comprises a non-digestible oligosaccharide. The non-digestible oligosaccharide preferably stimulates the growth of the beneficial intestinal bacteria, particularly lactic acid producing bacteria and/or bacteroides. The presence of non-digestible oligosaccharides acts synergistically with the inactivated bacteria and/or bacterial cell fragments by stimulating the growth of beneficial bacteria, particularly lactic acid bacteria and/or bacteroides, by reducing the growth of adverse bacteria in the intestinal tract and/or by directly advantageously stimulating the immune system. Hence, the presence of non-digestible oligosaccharides together with the inactivated bacteria and/or bacterial cell fragments advantageously results in both a faster and a higher colonization. Preferably the composition comprises at least two different non-digestible oligosaccharides. The presence of at least two different non-digestible oligosaccharides results in a microbiota more diverse in respect of different bacteria species, such as is the case in vaginally born infants. The presence of at least two different non-digestible oligosaccharides and the inactivated bacteria and/or bacterial cell fragments advantageously results in a faster, as well as a higher as well as a more diverse colonisation.

The term “oligosaccharide” as used in the present invention refers to saccharides with a degree of polymerization (DP) of 2 to 250, preferably a DP 2 to 100, more preferably 2 to 60, even more preferably 2 to 10. The term “non-digestible oligosaccharide” as used in the present invention refers to oligosaccharides which are not digested in the intestine by the action of acids or digestive enzymes present in the human upper digestive tract (small intestine and stomach) but which are preferably fermented by the human intestinal flora. For example, sucrose, lactose, maltose and maltodextrins are considered digestible.

The non-digestible carbohydrate are at least one, more preferably at least two selected from the group consisting of fructo-oligosaccharides, galacto-oligosaccharides, gluco-oligosaccharides, arabino-oligosaccharides, mannan-oligosaccharides, xylo-oligosaccharides, fuco-oligosaccharides, arabinogalacto-oligosaccharides, glucomanno-oligosaccharides, galactomanno-oligosaccharides sialic acid comprising oligosaccharides and uronic acid oligosaccharides. Preferably the present composition comprises fructo-oligosaccharides, galacto-oligosaccharides and/or galacturonic acid oligosaccharides, more preferably galacto-oligosaccharides, most preferably beta-galacto-oligosaccharides. The group of fructo-oligosaccharides includes inulin, the group of galacto-oligosaccharides includes transgalacto-oligosaccharides or beta-galacto-oligosaccharides, the group of gluco-oligosaccharides includes gentio-, nigero- and cyclodextrin-oligosaccharides and polydextrose, the group of arabinogalacto-oligosaccharides includes gum acacia, and the group of galactomanno-oligosaccharides includes partially hydrolysed guar gum.

The present composition preferably comprises at least two non-digestible oligosaccharides with different average degrees of polymerization (DP). For further improvement, the present non-digestible oligosaccharide preferably has a relatively high content of short chain oligosaccharides, as these strongly stimulate the growth of bifidobacteria.

Preferably the composition comprises galacto-oligosaccharides. The galacto-oligosaccharides are preferably selected from the group consisting of beta-galacto-oligosaccharides, lacto-N-tetraose (LNT), lacto-N-neotetraose (neo-LNT), fucosyl-lactose, fucosylated LNT and fucosylated neo-LNT. In a particularly preferred embodiment the present composition comprises beta-galacto-oligosaccharides. Beta-galacto-oligosaccharides as used in the present invention refers to oligosaccharides composed of over 50%, preferably over 65% galactose units based on monomeric subunits, with a degree of polymerization (DP) of 2 to 20, in which at least 50%, more preferably at least 75%, even more preferably at least 90%, of the galactose units are linked together via a beta-glycosidic linkage, preferably a beta-1,4 or beta 1,6 glycosidic linkage. Beta-linkages are also predominant in human milk oligosaccharides. The average DP is preferably of 3 to 6. A glucose unit may be present at the reducing end of the chain of galactose units. Beta-galacto-oligosaccharides are sometimes also referred to as transgalacto-oligosaccharides (TOS). A suitable source of beta-galacto-oligosaccharides is Vivinal®GOS (commercially available from Borculo Domo Ingredients, Zwolle, Netherlands). Other suitable sources are Oligomate (Yakult), Cupoligo, (Nissin) and Bi2muno (Classado). Beta-galacto-oligosaccharides were found to be most effective in stimulating the growth of lactic acid bacteria, preferably bifidobacteria.

Preferably the composition comprises fructo-oligosaccharides. Fructo-oligosaccharides as used in the present invention refers to carbohydrates composed of over 50%, preferably over 65% fructose units based on monomeric subunits, in which at least 50%, more preferably at least 75%, even more preferably at least 90%, of the fructose units are linked together via a beta-glycosidic linkage, preferably a beta-2,1 glycosidic linkage. A glucose unit may be present at the reducing end of the chain of galactose units. Preferably the fructo-oligosaccharide has a DP or average DP of 2 to 250, more preferably 2 to 100, even more preferably 10 to 60. Fructo-oligosaccaride comprises levan, hydrolysed levan, inulin, hydrolysed inulin, and synthesised fructo-oligosaccharides. Preferably the composition comprises short chain fructo-oligosaccharides with an average degree of polymerization (DP) of 3 to 6, more preferably hydrolysed inulin or synthetic fructo-oligosaccharide. Preferably the composition comprises long chain fructo-oligosaccharides with an average DP above 20, such as RaftilinHP. Preferably the composition comprises both short chain and long chain fructo-oligosaccharides. Fructo-oligosaccharide suitable for use in the compositions is also readily commercially available, e.g. RaftilineHP and RaftiloseP95(Orafti).

More preferably the composition comprises a combination of galacto-oligosaccharides and fructo-oligosaccharides, more preferably long chain fructo-oligosaccharides. Such a mixture stimulates the growth of a healthy intestinal flora, particularly bifidobacteria and/or lactobacilli and reduces the occurrence of E. coli in infants delivered via caesarean section. The mixture synergistically stimulates lactic acid bacteria, in particular bifidobacteria.

The present composition preferably comprises uronic acid oligosaccharides, more preferably galacturonic acid oligosaccharides. The term uronic acid oligosaccharide as used in the present invention refers to an oligosaccharide wherein at least 50% of the monosaccharide units present in the oligosaccharide is uronic acid. The term galacturonic acid oligosaccharide as used in the present invention refers to an oligosaccharide wherein at least 50% of the monosaccharide units present in the oligosaccharide is galacturonic acid. The galacturonic acid oligosaccharides used in the invention are preferably prepared from degradation of pectin, pectate, and/or polygalacturonic acid. Preferably the degraded pectin is prepared by hydrolysis and/or beta-elimination of fruit and/or vegetable pectins, more preferably apple, citrus and/or sugar beet pectin, even more preferably apple, citrus and/or sugar beet pectin degraded by at least one lyase. In a preferred embodiment, at least one of the terminal uronic acid units of the uronic acid oligosaccharide has a double bond. Preferably one of the terminal uronic acid units comprises a C₄-C₅ double bond. The uronic acid oligosaccharide can be derivatised. The uronic acid oligosaccharide may be methoxylated and/or amidated. Preferably the uronic acid oligosaccharides are characterised by a degree of methoxylation above 20%, preferably above 50% even more preferably above 70%. The double bond effectively protects against attachment of pathogenic bacteria to intestinal epithelial cells, thereby reducing colonization of (nosocomial) pathogenic bacteria in the colon of the infant delivered by caesarean section. Furthermore, uronic acid oligosaccharides preferably stimulate the formation of a healthy intestinal flora and are fermented, resulting in a production of intestinal organic acids and a reduction of intestinal pH, which inhibit the growth of (nosocomial) pathogenic bacteria.

Thus, in one embodiment the composition for use according to the present invention preferably comprises at least beta-galacto-oligosaccharides. In one embodiment the composition for use according to the present invention preferably comprises at least short chain fructo-oligosaccharides and/or long chain fructo-oligosaccharides, preferably long chain fructo-oligosaccharides. In one embodiment the composition for use according to the present invention preferably comprises at least uronic acid oligosaccharides. In one embodiment the composition for use according to the present invention preferably comprises at least beta-galacto-oligosaccharides and at least short chain fructo-oligosaccharides or long chain fructo-oligosaccharides or both. In one embodiment the composition for use according to the present invention preferably comprises at least beta-galacto-oligosaccharides and at least uronic acid oligosaccharides. In one embodiment the composition for use according to the present invention preferably comprises at least short chain fructo-oligosaccharides and uronic acid oligosaccharides or long chain fructo-oligosaccharides and uronic acid oligosaccharides. In one embodiment the composition for use according to the present invention preferably comprises at least beta-galacto-oligosaccharides and short chain fructo-oligosaccharides and uronic acid oligosaccharides or at least beta-galacto-oligosaccharides and long chain fructo-oligosaccharides and uronic acid oligosaccharides. Preferably the weight ratio between the mixture of two different non-digestible carbohydrates, preferably beta-galacto-oligosaccharides and fructo-oligosaccharide, is between 20 and 0.05, more preferably between 20 and 1. Beta-galacto-oligosaccharides are more reminiscent to the human milk oligosaccharides. Preferably the present composition comprises beta-galacto-oligosaccharides with a DP of 2-10 and/or fructo-oligosaccharides with a DP of 2-60. This combination was found to synergistically increase bifidobacteria and lactobacilli. The presence of these three non-digestible oligosaccharides even further stimulates the bifidobacteria. The weight ratio transgalacto-oligosaccharide: fructo-oligosaccharide: pectin degradation product is preferably (20 to 2):1:(1 to 20), more preferably (12 to 7):1:(1 to 3).

Preferably, the composition comprises of 80 mg to 3 g non-digestible oligosaccharides per 100 ml, more preferably 150 mg to 2 g, even more preferably 300 mg to 1.5 g. Based on dry weight, the composition preferably comprises 0.05 wt. % to 75 wt. %, more preferably 0.1 wt. % to 20 wt. %, even more preferably 0.5 wt. % to 10 wt. %. A lower amount of non-digestible oligosaccharides will be less effective in stimulating the beneficial bacteria in the microbiota, whereas a too high amount will result in side-effects of bloating and abdominal discomfort.

### Formulae

The composition used in the present invention are enteral nutritional compositions and suitable for administration to caesarean section delivered infants. The present composition is enterally administered, more preferably orally.

The present composition is preferably an infant formula. The present composition can be advantageously applied as a complete nutrition for infants. The present composition preferably comprises lipid, protein and digestible carbohydrate and is preferably administered in liquid form. The present invention includes dry food (e.g. powders) which is accompanied with instructions as to mix said dry food mixture with a suitable liquid (e.g. water).

The present invention advantageously provides a composition wherein the fat provides 5 to 50% of the total calories, the protein provides 5 to 50% of the total calories, and the digestible carbohydrate component provides 15 to 90% of the total calories. In one embodiment the composition comprises protein, fat and digestible carbohydrate, wherein the protein provides 5 to 25% of the total calories, the fat provides 25 to 60% of the total calories, and the digestible carbohydrate provides 30 to 70% of the total calories. Preferably, in the present composition the lipid provides 35 to 50% of the total calories, the protein provides 7.5 to 12.5% of the total calories, and the digestible carbohydrate provides 40 to 55% of the total calories. For calculation of the % of total calories for the protein, the total of energy provided by proteins, peptides and amino acids needs to be taken into account.

The present composition preferably comprises at least one lipid selected from the group consisting of animal lipid (excluding human lipids) and vegetable lipids. Preferably the present composition comprises a combination of vegetable lipids and at least one oil selected from the group consisting of fish oil, animal oil, algae oil, fungal oil, and bacterial oil. The present composition excludes human milk.

The protein used in the nutritional preparation is preferably selected from the group consisting of non-human animal proteins (preferably milk proteins), vegetable proteins (preferably soy protein and/or rice protein), hydrolysates thereof, free amino acids and mixtures thereof. The present composition preferably contains casein, whey, hydrolysed casein and/or hydrolysed whey protein. Preferably the protein comprises intact proteins, more preferably intact bovine whey proteins and/or intact bovine casein proteins. As the present composition is suitably used to reduce the allergic reaction in an infant, the protein of is preferably selected from the group consisting of hydrolyzed milk protein. Preferably the present composition comprises hydrolyzed casein and/or hydrolyzed whey protein, vegetable protein and/or amino acids. The use of these proteins further reduced the allergic reactions of the infant. The use of these hydrolysed proteins advantageously improves the absorption of the dietary protein component by the immature intestine of the infant delivered by caesarean section.

The present composition preferably comprises digestible carbohydrates selected from the group consisting of sucrose, lactose, glucose, fructose, corn syrup solids, starch and maltodextrins, more preferably lactose.

The present composition preferably has a viscosity between 1 and 60 mPa·s, preferably between 1 and 20 mPa·s, more preferably between 1 and 10 mPa·s, most preferably between 1 and 6 mPa·s. The low viscosity ensures a proper administration of the liquid, e.g. a proper passage through the whole of a nipple. Also this viscosity closely resembles the viscosity of human milk. Furthermore, a low viscosity results in a normal gastric emptying and a better energy intake, which is essential for infants which need the energy for optimal growth and development. The present composition is preferably prepared by admixing a powdered composition comprising with water. Normally infant formula is prepared in such way. The present invention thus also relates to a packaged power composition wherein said package is provided with instruction to admix the powder with a suitable amount of liquid, thereby resulting in a liquid composition with a viscosity between 1 and 60 mPa·s. The viscosity of the liquid is determined using a Physica Rheometer MCR 300 (Physica Messtechnik GmbH, Ostfilden, Germany) at a shear rate of 95 s⁻¹ at 20° C.

Stool irregularities (e.g. hard stools, insufficient stool volume, and diarrhoea) are an important problem in babies delivered via caesarean section. This may be caused by the high content of E. coli in the faeces. It was found that stool problems may be reduced by administering the present non-digestible oligosaccharides in liquid food with an osmolality between 50 and 500 mOsm/kg, more preferably between 100 and 400 mOsm/kg. The reduced stool irregularities enhance the colonization and development of a healthy intestinal microbiota.

In view of the above, it is also important that the liquid food does not have an excessive caloric density, however still provides sufficient calories to feed the subject. Hence, the liquid food preferably has a caloric density between 0.1 and 2.5 kcal/ml, even more preferably a caloric density of between 0.5 and 1.5 kcal/ml, most preferably between 0.6 and 0.8 kcal/ml.

In one embodiment the present invention relates to a sachet containing a composition to be administered to a caesarean delivered infant comprising inactivated Gram-negative bacteria and/or bacterial cell fragments of Gram-negative bacteria and optionally Gram-positive bacteria and/or bacterial cell fragments of Gram-positive bacteria wherein the composition comprises less than 10³ cfu Gram-negative bacteria, more preferably less than 10³ cfu bacteria, per g dry weight. Preferably the sachet comprises inactivated bacteria or cell fragments obtained from more than 1×10² cfu, in particular at least 1×10³ Gram-positive plus Gram-negative, more preferably Gram-negative bacteria, per g based on dry weight of the final composition, more preferably more than 1×10⁴ cfu, even more preferably more than 1×10⁶ cfu. Preferably the sachet comprises inactivated bacteria or cell fragments obtained from less than 1×10¹² cfu

Gram-positive and Gram-negative bacteria, more preferably Gram-negative bacteria, per g based on dry weight of the final composition, more preferably 1×10¹⁰ cfu, even more preferably 1×10⁹ cfu. Preferably the sachet additionally comprises non-digestible oligosaccharides, more preferably at least two, non-digestible oligosaccharides selected from the group consisting of fructo-oligosaccharides, galacto-oligosaccharides, gluco-oligosaccharides, arabino-oligosaccharides, mannan-oligosaccharides, xylo-oligosaccharides, fuco-oligosaccharides, arabinogalacto-oligosaccharides, glucomanno-oligosaccharides, galactomanno-oligosaccharides, sialic acid comprising oligosaccharides and uronic acid oligosaccharides. Preferably the sachet contains 0.25 to 5 g, more preferably 0.5 to 2 g non-digestible oligosaccharides.

### Application

The present invention provides in one embodiment an enteral nutrional composition for use in administration to caesarean delivered infants. Preferably, the present invention provides (i) the treatment and/or prevention of a disorder in infants delivered via caesarean section and/or (ii) the stimulation of health in infants delivered via caesarean section. The disorder is preferably selected from a group consisting of intestinal disorders caused by a microbiota low in bifidobacteria and/or bacteroides. Preferably the disorder is selected from the group of allergy, eczema, asthma, infection and diarrhoea.

In one aspect the present invention provides a nutritional composition according to the present invention for use in the treatment of a disorder selected from the group consisting of allergy, eczema, asthma, infection and diarrhoea.

Providing the new-born caesarean section delivered infant with immunogenic factors of Gram-negative bacteria and preferably also Gram-positive bacteria (e.g. inactivated cells and/or bacteria cell envelop fragments such as glycoproteins, glycolipids, peptidoglycan, lipopolysaccharides (LPS), lipoteichoic acid (LTA), flagellae, lipoproteins, capsular polysaccharides and/or DNA) induces tolerance for those bacteria, thereby increasing intestinal colonization for these bacteria, and/or may decrease the colonization of adverse bacteria. These immunogenic factors may also have a direct effect on stimulating the growth of bifidobacteria and/or decreasing the growth of adverse bacteria. Furthermore, the presence of inactivated Gram-negative bacteria and/or cell fragments form Gram-negative bacteria additionally has the advantage of resulting in an increased immunity against infections, intestinal infections as well as systemic infections, with Gram-negative bacteria. Induction of tolerance against bacteria in the intestinal tract results in a faster colonisation by the desired bacteria, while on the other hand the absence of living cells in the product results in an increased safety and improved product technological properties. The safety advantage is especially important in case of Gram-negative bacteria, which are generally not recognized as safe.

The present invention preferably provides a method for the prevention and/or treatment of infections and/or infection disorders, particularly gastrointestinal infections, more preferably the treatment and/or prevention of infections caused by one or more micro-organisms selected from the group consisting of Staphylococcus (especially S. aureus, S. epidermidis, S. haemolyticus), Streptococcus (especially Streptococcus group B), Clostridium (especially C. difficile), Bacillus (especially B. subtilis, Pseudomonas (especially P. aeruginosa), Enterobacter, Klebsiella, Acinetobacter, Proteus, Aeromonas, and Escherichia, preferably Escherichia coli (E. coli) said method comprising administering a nutritional composition according to the present invention.

Preferably, the present composition is used in a method for treatment and/or prevention of intestinal infection, intestinal inflammation and/or diarrhoea in infants delivered by caesarean section. Preferably the present composition is used in a method for modulating the immune system in infants born via caesarean section. In a further aspect, the present invention therefore provides a method for treatment and/or prevention of systemic infections, urinary tract infections, otitis and/or respiratory infections in infants delivered by caesarean section, said method comprising administering a nutritional composition according to the present invention.

In a further aspect, the present invention provides a method for treatment and/or prevention of allergy (particularly food allergy, more particularly cow's milk allergy), atopic eczema (e.g. atopic dermatitis), asthma, allergic rhinitis, and allergic conjunctivitis, even more preferably allergy and/or asthma, in infants, preferably in infants delivered by caesarean section, said method comprising administering to the infant a composition comprising inactivated Gram-negative bacteria and/or bacterial cell fragments of Gram-negative bacteria and optionally Gram-positive bacteria, which are preferably inactivated, and/or bacterial cell fragments of Gram-positive bacteria. These health effects are obtained by effects on immune system and/or intestinal microbiotica.

Administration of the present composition results in an improved intestinal microbiota and subsequently in the formation of organic acids as metabolic end products of microbial fermentation. An increased amount of organic acids results in an increased mucus production, improves gut maturation and/or and increased gut barrier. Hence, in a further aspect, the present invention provides a method for decreasing intestinal wall permeability in caesarean section delivered infants and/or for improving intestinal wall maturation in caesarean section delivered infants, said method comprising administering to the infant a composition comprising the inactivated Gram-negative bacteria and/or bacteria cell fragments of Gram-negative bacteria and optionally Gram-positive bacteria, which are preferably inactivated, and/or bacterial cell fragments of Gram-positive bacteria.

Preferably the present composition is used for tolerance induction in the caesarean section delivered infant's intestine against bacteria and/or for improving intestinal colonisation of the microbiota in caesarean delivered infants towards the microbiota found in vaginally delivered infants and/or for a fast colonisation of a microbiota rich in lactic acid producing bacteria in caesarean section delivered infants.

In one embodiment the present invention relates to a method for the manufacture of an infant nutrition suitable for infants born via cesarean section comprising admixing human breast milk and a composition comprising inactivated cells of Gram-negative bacteria and/or bacterial cell fragments of Gram-negative bacteria and optionally cells of Gram-positive bacteria, which are preferably inactivated, and/or bacterial cell fragments of Gram-positive bacteria, wherein the composition comprises less than 10³ cfu Gram-negative bacteria, more preferably less than 10³ cfu bacteria per g dry weight.

In one embodiment the present invention relates to a method for intestinal tolerance induction against bacteria in caesarean section delivered infants, and/or improving the colonisation of the intestinal microbiota in caesarean delivered infants comprising the step of admixing of i) a nutritionally or pharmaceutically acceptable liquid; and ii) a dry composition, wherein the dry composition comprises inactivated Gram-negative bacteria and/or bacterial cell fragments of Gram-negative bacteria and optionally Gram-positive bacteria, which are preferably inactivated, and/or bacterial cell fragments of Gram-positive bacteria and wherein the composition comprises less than 10³ cfu Gram-negative bacteria per g dry weight, preferably less than 10³ cfu bacteria per g dry weight, and the step of administering the composition obtained in the first step to the infant.

In other words the invention concerns a composition obtained by admixing i) a nutritionally or pharmaceutically acceptable liquid; and ii) a dry composition, wherein the dry composition comprises inactivated Gram-negative bacteria and/or bacterial cell fragments of Gram-negative bacteria and optionally Gram-positive bacteria, which are preferably inactivated, and/or bacterial cell fragments of Gram-positive bacteria, wherein the dry composition comprises less than 10³ cfu Gram-negative bacteria per g dry weight, for use for intestinal tolerance induction against bacteria in infants delivered via caesarean section, and/or improving the colonisation of the intestinal microbiota in infants delivered via caesarean section.

The invention can also be worded as the use of a composition obtained by admixing i) a nutritionally or pharmaceutically acceptable liquid; and ii) a dry composition, wherein the dry composition comprises inactivated Gram-negative bacteria and/or bacterial cell fragments of Gram-negative bacteria and optionally Gram-positive bacteria, which are preferably inactivated, and/or bacterial cell fragments of Gram-positive bacteria, wherein the dry composition comprises less than 10³ cfu Gram-negative bacteria per g dry weight, for use in the manufacture of a nutritional composition for intestinal tolerance induction against bacteria in infants delivered via caesarean section, and/or improving the colonisation of the intestinal microbiota in infants delivered via caesarean section.

The present composition is preferably administered to the infant delivered via caesarean section in the first year of life, preferably within 3 months after birth, more preferably within six weeks after birth, even more preferably within two weeks after birth, even more preferably within one week after birth, more preferably within 72 hours, most preferably within 48 hours after birth.

### EXAMPLES

### Example 1

### Molecular Characterization of Intestinal Microbiota in Infants Born by Vaginal delivery vs. Caesarean Delivery

In the present study the influence of mode of delivery (caesarean delivery versus vaginal delivery) on the intestinal microbial composition at the third day of life by was studied using by PCR amplification with species-specific primers for one Bacteroides species and E. coli.

The microbial DNA was extracted and analyzed according to Favier et al, Environ Microbiol 2002; 68:219-226 and Satokari et al, Appl Environ Microbiol 2001; 67:504-513; Satorkari et al System Appl Microbiol 2003; 26:572-584.

The results of bacterial species detected in faecal samples of 23 newborns after caesarean delivery obtained at the 3rd day of life and of 23 newborns after vaginal delivery obtained at the 3rd day of life are as follows:

E. coli was found in 9 of 23 (39.1%) vaginally delivered newborns, whereas it was found only in 2 of 23 (8.7%) cesarean-delivered newborns.

Bacteroides has been found in 8.7% of vaginally delivered babies only, and was absent in caesarean delivered infants.

It can be concluded that the microbial flora of an infant born via caesarean section differs from that of an infant born via the vaginal route by having a lower amount of Gram-negative bacteria.

These results are indicative for the advantageous use of the composition and method according to the present invention.

### Example 2

### Composition for Babies Born Via Caesarean Section

An infant formula comprising protein 21 g/l, fat 24 g/l, carbohydrates 83 g/l, non-digestible oligosaccharides 8 g/l, minerals 5 g/l, vitamins 0.45 g/l was prepared.

Fat was added to UHT sterilised milk at 70° C. and the mixture was homogenised in two stages, the first one at 200 kg/cm², the second at 50 kg/cm². At 37° C. the mixture was inoculated with 1.5% of a culture of B. breve 1-2219 containing 1 to 5×10⁹ bacteria/ml and incubated for 8 h at 37° C. The mixture was then cooled to 5° C. The rest of the ingredients was dissolved in water and added to the resulting product. The composition comprised per 100 ml ready to drink formula 0.72 g beta-galacto-oligosaccharides and 0.08 g long chain and/or short chain inulin.

Additionally 250 ml of heat killed Bacteroides fragilis are added. The heat killed B. fragilis were obtained by growing B. fragilis in 250 ml of the chemically defined medium of Varel & Bryant, 1974, Appl Microbiol 18:251-257 to a cell density of 7×10⁸ bacteria/ml and subsequent sterilization of the culture.

The resulting mixture was UHT sterilised at 140° C. for 6 to 7 seconds and aseptically packed.

### Example 3

### Composition for Babies born Via Caesarean Section

An infant formula was prepared comprising per 100 g dry material: protein (80% casein and 20% whey) 13 g; vegetable fat 25.5 g; lactose 42.25 g; maltodextrin 16 g; minerals 3 g; vitamins 0.25 g.

Vegetable fat was added to cow's milk heated at 75° C. The mixture was homogenised in two stages, the first one at 200 kg·s/cm2, the second at 50 kg·s/cm². Aqueous solutions of lactose and maltodextrin and vitamins and minerals were added. The composition was pasteurised at 115° C. and concentrated by evaporation to 48% dry material. The concentrate was cooled to 37° C. and inoculated with 5% of a culture of B.breve 1-2219 containing 10⁹ bacteria/ml and incubated for 8 h at 37° C.

Additionally 250 ml of heat killed Bacteroides fragilis are added. The heat killed B. fragilis are obtained by growing B. fragilis in 250 ml of the chemically defined medium of Varel & Bryant, 1974, Appl Microbiol 18:251-257 to a cell density of 7×10⁸ bacteria/ml and subsequent sterilization of the culture. Subsequently the concentrate was pasteurized again.

The concentrate was spray dried and adding 140 g per litre water provided a reconstituted infant milk formula. Non-digestible oligosaccharides were included to result in 0.72 g beta-galacto-oligosaccharides and 0.08 g inulin per 100 ml ready to drink formula. The package and/or supporting material accompanying the product indicated that the product could suitably be used to a) stimulate intestinal colonisation with beneficial bacteria, b) prevent and/or treat infection in infants delivered via caesarean section; and/or c) prevent and/or treat allergy in infants delivered via caesarean section.

1-17. (canceled)

18. A nutritional composition comprising inactivated Gram-negative bacteria and/or bacterial cell fragments of Gram-negative bacteria and optionally Gram-positive bacteria and/or bacterial cell fragments of Gram-positive bacteria, wherein the composition comprises less than 103 cfu bacteria per g dry weight of the composition.

19. The composition according to claim 18, comprising inactivated Gram-positive bacteria and/or bacterial cell fragments of Gram-positive bacteria.

20. The composition according to claim 18, wherein the Gram-positive bacteria comprise lactic acid producing bacteria.

21. The composition according to claim 20, wherein the Gram-positive bacteria comprise bifidobacteria, lactobacilli and/or streptococci.

22. The composition according to claim 21, wherein the bifidobacteria comprise Bifidobacterium breve.

23. The composition according to claim 1, wherein the Gram-negative bacteria comprise bacteroides.

24. The composition according to claim 18, further comprising at least one non-digestible oligosaccharide selected from the group consisting of fructo-oligosaccharides, galacto-oligosaccharides, gluco-oligosaccharides, arabino-oligosaccharides, mannan-oligosaccharides, xylo-oligosaccharides, fuco-oligosaccharides, arabinogalacto-oligosaccharides, glucomanno-oligosaccharides, galactomanno-oligosaccharides, sialic acid comprising oligosaccharides and uronic acid oligosaccharides.

25. The composition according to claim 23, comprising galacto-oligosaccharidcs and/or fructo-oligosaccharides.

26. The composition according to claim 24, comprising 0.5 to 75 g of non-digestible oligosaccharide per 100 g dry weight of the composition.

27. The composition according to claim 18, further comprising, as a percentage of total calories of the composition:
(a) 5 to 25% calories from protein,
(b) 25 to 60% calories from fat, and
(c) 30 to 70% calories from carbohydrate.

28. A method of treatment and/or prevention of a disorder in infants delivered and/or the stimulation of health in infants, comprising administering to an infant delivered via caesarean section and in need thereof a composition according to claim 18.

29. The method according to claim 28, wherein the composition is enterally administered.

30. The method according to claim 28, wherein the composition is administered within one week after birth of the infant.

31. The method according to claim 28, wherein the disorder is selected from the group consisting of allergy, eczema, asthma, infection and diarrhoea.

32. A method of inducing intestinal tolerance against bacteria and/or improving the colonisation of the intestinal microbiota in infants, comprising administering to an infant delivered via caesarean section and in need thereof a composition according to claim 18.

33. A method for the manufacture of an infant nutrition suitable for infants delivered via cesarean section comprising admixing:
(a) human breast milk; and
(b) a composition comprising inactivated Gram-negative bacteria and/or bacterial cell fragments of Gram-negative bacteria and optionally Gram-positive bacteria and/or bacterial cell fragments of Gram-positive bacteria,
wherein the composition comprises less than 103 cfu Gram-negative bacteria per g dry weight of the composition.

34. A sachet comprising a composition comprising inactivated Gram-negative bacteria and/or bacterial cell fragments of Gram-negative bacteria and optionally Gram-positive bacteria and/or bacterial cell fragments of Gram-positive bacteria, wherein the composition comprises less than 103 cfu Gram-negative bacteria per g dry weight.

35. A composition obtained by admixing
(a) a nutritionally or pharmaceutically acceptable liquid; and
(b) a dry composition, wherein the dry composition comprises
inactivated Gram-negative bacteria and/or bacterial cell fragments of Gram-negative bacteria and optionally Gram-positive bacteria and/or bacterial cell fragments of Gram-positive bacteria,
wherein the dry composition comprises less than 103 cfu Gram-negative bacteria per g dry weight.
